(19) United States
(12) Patent Application Publication
(10) Pub. No.: US 2026/0266797 A1
(43) Pub. Date: Sep. 10, 2026

(54) METHOD FOR QUANTIFYING THE PYROGENIC CARBON PRESENT IN A SAMPLE OF AN ORGANO-MINERAL OR MINERAL MATRIX COMPRISING CHARCOAL AND/OR BIOCHAR

(71) Applicant: IFP Energies nouvelles, RUEIL-MALMAISON (FR)

(72) Inventors: David SEBAG, RUEIL-MALMAISON CEDEX (FR); Marie-Liesse AUBERTIN, RUEIL-MALMAISON CEDEX (FR)

(21) Appl. No.: 19/167,473

(22) PCT Filed: Mar. 15, 2024
(86) PCT No.: PCT/EP2024/057084

§ 371 Date: Sep. 22, 2025

(30) Foreign Application Priority Data
Apr. 6, 2023 (FR) FR2303436

(51) Int. Cl. G01N 33/24; G01N 31/00

(52) U.S. Cl. CPC G01N 33/24; G01N 31/005

(57) ABSTRACT

The present invention is a process for quantifying pyrogenic carbon content of a sample comprising an organo-mineral or mineral matrix and also at least one of the charcoal and biochar. The process involves heating the sample under an inert atmosphere, followed by heating under an oxidizing atmosphere of the residue from the heating under an inert atmosphere, and measuring the amount of CO₂ released during the heating under an oxidizing atmosphere. The measured curve is broken down into a first component corresponding to the part of the curve associated with temperatures below a limit temperature, and a second component corresponding to a part of the curve associated with temperatures above or equal to the limit temperature. The limit temperature is between 500° C. and 550° C. The pyrogenic carbon content is determined from the surface areas of the first and second components.

### CROSS-REFERENCE TO RELATED APPLICATIONS

This application is a 371 National Phase application under 35 U.S.C. § 371 of PCT/EP2024/057084 filed Mar. 15, 2024, and French Application No. 2303436, filed Apr. 6, 2023, which are incorporated herein by reference in their entireties.

### BACKGROUND OF THE INVENTION

### Field of the Invention

The present invention may relate in particular to the field of soil science, agronomy and the environment, and more generally to any field concerned with the quantification of at least one biochar and/or charcoal present in a mineral or an organo-mineral matrix.

The present invention may, for example, relate to the field of biochar production and marketing, in particular when sold in the form of a mixture with an organo-mineral or mineral matrix, for use, for example, as an organic soil improver (biochar-sediment) or as a building material, like hybrid green concrete (biochar-calcium carbonate) or simply by mixing biochar with concrete (mineral matrix).

### Description of the Prior Art

In the environmental field, in order to meet environmental challenges and notably to limit global warming to 1.5° C. compared to pre-industrial times, it is necessary to achieve the “Net Zero Emissions” objective by 2050. To achieve this, it is necessary not only to drastically reduce CO₂ emissions, but also to develop negative emission technologies. Biochar, a product of biomass pyrolysis, constitutes a negative emission technology, as it is a carbon-rich, biologically stable material. Moreover, in recent decades, pyrogenic organic matter has been recognized as an important component of very fertile Amazonian soil. Specifically, this material may have beneficial effects on soil fertility, in particular for water and nutrient retention and a liming effect. The name given to pyrolyzed organic matter differs as a function of its end use. The term “biochar” is used when the organic matter is pyrolyzed with the object of amending the soil to improve its properties. In the literature, “black carbon” describes the volatile components resulting from the incomplete combustion of biomass. In certain studies, the term “black carbon” is also used to describe a wider range of materials, forming a continuum ranging from charcoal to soot. The term “char” or “charcoal” or sometimes “wood charcoal” is used when organic matter is deliberately pyrolyzed, for example for use as a fuel or filter. It should be noted that charcoal differs from geological coal or coal of geological origin in the way it is produced, and also in its very different chemical and physical properties. Unlike charcoal derived from the pyrolysis of biomass, geological coal (for example hard coal) is produced by geological processes under high pressure and high temperature, over a longer timescale (several thousand years). Geological coal notably does not contain any pyrogenic carbon.

Thus, forms of charcoal other than geological coal (i.e. charcoal, char or biochar) may be present in a soil due to several contexts, such as vegetation fires and intentional amendment to improve soil properties. As charcoal is rich in biologically stable carbon (i.e. aromatic compounds), its quantification can be carried out through the pyrogenic carbon of which it is predominantly formed. There are many advantages to being able to quantify pyrogenic carbon in soil, depending on the context in which it has been introduced. In the case of vegetation fires, the pyrogenic carbon content constitutes a marker of fire frequency. This marker is particularly useful for archaeological studies, since high fire frequencies on the same site may attest to previous human occupation, while lower intensities are more likely to be the result of fires of natural origin. In the case of biochar amendments, quantifying the pyrogenic carbon in an amended plot enables monitoring of the remaining biochar content in the soil. Specifically, biochar may be readily subject to physical degradation. Moreover, quantifying the pyrogenic carbon outside the amended plot allows monitoring of the biochar's fate in the environment, notably its deposition zones.

The following documents will be mentioned in the course of the description:
Arroyo-Kalin, M. A. (2008). Steps Towards an Ecology of Landscape: A Geoarchaeological Approach to the Study of Anthropogenic Dark Earths in the Central Amazon Region, Brazil (Doctoral dissertation, University of Cambridge).
Aubertin, M. L. (2022). Biochar-Compost Mixtures: Interactions and Impact on Carbon Sequestration and Soil Fertility (Doctoral dissertation, Sorbonne University).
Behar, F., Beaumont, V., & Penteado, H. D. B. (2001). Rock-Eval 6 Technology: Performances and Developments. Oil & Gas Science and Technology, 56(2), 111-134.
Chalk, P., & Smith, C. J. (2022). 13C Methodologies for Quantifying Biochar Stability in Soil: A critique. European Journal of Soil Science, 73(3), e13245.
Cuypers, C., Grotenhuis, T., Nierop, K. G., Franco, E. M., de Jager, A., & Rulkens, W. (2002). Amorphous and Condensed Organic Matter Domains: the Effect of Persulfate Oxidation on the Composition of Soil/Sediment Organic Matter. Chemosphere, 48(9), 919-931.
Glaser, B., Haumaier, L., Guggenberger, G., & Zech, W. (1998). Black Carbon in Soils: the use of Benzenecarboxylic Acids as Specific Markers. Organic Geochemistry, 29(4), 811-819.
Llorente, M., Turrión, M. B., & Glaser, B. (2018). Rapid and Economical Quantification of Black Carbon in Soils using a Modified Benzene Polycarboxylic Acids (BPCA) Method. Organic Geochemistry, 115, 197-204.
Paterson, G. A., & Heslop, D. (2015). New Methods for Unmixing Sediment Grain Size data. Geochemistry, Geophysics, Geosystems, 16(12), 4494-4506.
Poot, A., Quik, J. T., Veld, H., & Koelmans, A. A. (2009). Quantification Methods of Black Carbon: Comparison of Rock-Eval Analysis with Traditional Methods. Journal of Chromatography A, 1216(3), 613-622.
Sebag, D., Disnar, J. R., Guillet, B., Di Giovanni, C., Verrecchia, E. P., & Durand, A. (2006). Monitoring Organic Matter Dynamics in Soil Profiles by “Rock-Eval Pyrolysis”: Bulk Characterization and Quantification of Degradation. European Journal of Soil Science, 57(3), 344-355.
Sebag, D., Garcin, Y., Adatte, T., Deschamps, P., Ménot, G., & Verrecchia, E. P. (2018). Correction for the Siderite Effect on Rock-Eval Parameters: Application to the Sediments of Lake Barombi (southwest Cameroon). Organic Geochemistry, 123, 126-135.
Simpson, M. J., & Hatcher, P. G. (2004). Overestimates of Black Carbon in Soils and Sediments. Naturwissenschaften, 91(9), 436-440.

Various techniques are known for quantifying pyrogenic carbon in soil or sedimentary material, based on chemical, magnetic, optical or thermal differences between soil and charcoal, or on the presence of molecular markers.

A commonly used technique is quantification by extraction of benzene polycarboxylic acids (BPCA) following chemical oxidation of aromatic structures, by gas chromatographic analysis, as described for example in (Glaser et al., 1998), or else by elemental analysis, as described for example in (Llorente et al. 2018). However, extraction is time-consuming and may add bias to BPCA quantification.

The use of natural carbon isotopy is a direct, accurate and reproducible quantification method, allowing the source of a carbon from a mixture of two carbon components with significantly different isotopic (δ13C) signatures to be distinguished, as described for example in (Aubertin et al., 2022). However, isotopic analysis may only be applied in the case of a charcoal-soil mixture, in which the two components have significantly different isotopic signatures. Isotopic enrichment methods can also be used to quantify pyrogenic carbon, but this involves (time-consuming) incubation and the results may be biased by the nonuniform distribution of labeled carbon added to the plant, as described for example in (Chalk and Smith, 2022).

Other methods for quantifying charcoal are based on the observation of differences in color or density of charcoal particles from a microscope photograph, as described for example in (Arroyo-Kalin, 2008). However, these methods are time-consuming (preparation time for thin slides and sample handling), only take into account particles above a certain diameter, are not very reproducible as they are manipulator-dependent, and only approximate the mass of the charcoal, based on a surface count.

Among thermal methods, the thermochemical oxidation method involves a chemical oxidation pretreatment with an acid to remove inorganic carbon, then separation of pyrogenic and non-pyrogenic carbon with combustion at temperatures of about 350° C. for at least 2 hours, followed by residual carbon analysis by 13C NMR or elemental analysis, as described for example in (Poot et al. 2009). In addition to the time-consuming aspect of this method, it may also lead to overestimates of pyrogenic carbon due to the formation of pyrogenic carbon during the combustion phase, as described for example in (Simpson and Hatcher, 2004). Thermogravimetric analysis measures several emission peaks during heating, but it is nevertheless difficult to differentiate emission peaks related to pyrogenic carbon and soil components with this method, as described for example in (Cuypers et al., 2002). One method for quantifying pyrogenic carbon in soil is the differential scanning calorimetry (DSC) technique, in which the sample causes changes in the flow of heat as a function of the temperature rise gradient. A close correlation may be made between variations in the flow of heat and the amount of carbon. To quantify the pyrogenic carbon, it suffices to differentiate between the amount of carbon above a threshold temperature, around 400° C., in a sample of the same soil/sediment with and without carbon. However, DSC is an indirect measurement of stable carbon in a sample, which may introduce inaccuracy in carbon quantification.

Thus, despite its obvious interest, the quantification of pyrogenic carbon in soil often remains difficult to perform, and the various existing techniques have drawbacks due to, for example, their price, analysis time, accuracy, or the fact that they are not always reproducible.

Methods for the thermal analysis of the organic matter of soils which rely on measurements of amounts of hydrocarbon (HC) compounds, of carbon monoxide (CO) and/or of carbon dioxide (CO2) released over time by a sample subjected to a temperature sequence under an inert atmosphere (pyrolysis phase) and then/or to a temperature sequence under an oxidizing atmosphere (oxidation phase) are also known. These methods were initially developed in the field of the petroleum industry, for the purposes of characterizing the organic fraction of sedimentary rocks. For example, the “ROCK-EVAL® BULK ROCK” method, initially developed in the context of conventional source rock samples, is known to distinguish pyrolyzed organic carbon from refractory organic carbon (Behar et al., 2001). The document (Poot et al., 2009) describes that the amount of refractory carbon measured in such a thermal analysis can be used to approximate the quantification of pyrogenic carbon in a soil or sediment sample. More specifically, this document describes that the “ROCK-EVAL® BULK ROCK” method allows pyrolyzable carbon (PC) to be readily and quickly separated from residual carbon (RC). RC corresponds to refractory organic carbon, derived from organic matter that is thermally resistant to the pyrolysis phase and is oxidized during the oxidation phase. The document thus proposes to approximate RC as a measure of pyrogenic carbon, which it calls “black carbon” and defines as a continuum ranging from pyrolyzed biomass charcoal to soot. However, this latter variant remains imprecise, due to the fact that RC may also be partly produced during the pyrolysis phase. Thus, the pyrogenic carbon analyzed with this method may be slightly overestimated.

### SUMMARY OF THE INVENTION

The present invention allows these drawbacks to be overcome. In particular, the present invention allows rapid and accurate quantification of pyrogenic carbon in a sample of an organo-mineral or mineral matrix such as soil, by means of thermal analysis, notably comprising analysis of carbon emissions during an oxidation phase of the sample.

The present invention relates to a process for quantifying the pyrogenic carbon content present in a sample comprising an organo-mineral or mineral matrix and also at least one of charcoal and biochar. The process according to the invention comprises at least the following steps:
A) the sample is heated under an inert atmosphere according to a first temperature sequence, with an initial temperature (TO) of between 100 and 300° C., preferentially equal to 200° C., and a final temperature (TF) of between 500 and 800° C., preferentially equal to 650° C.;
B) a residue of the sample resulting from the heating under an inert atmosphere is heated under an oxidizing atmosphere according to a second temperature sequence with an initial temperature (T0′) of between 10° and 300° C., preferentially equal to 200° C., and a final temperature (TF′) of between 700 and 1000° C., preferentially equal to 850° C., and measuring at least one amount of CO₂ (QCO2) released during the second temperature sequence;
C) on the basis of a curve representing the temperature-dependent evolution of the quantity of CO₂ released during the heating in an oxidizing atmosphere, the curve is broken down into at least a first and a second component, the first component corresponding to a part of the curve associated with temperatures below a limit temperature, and the second component corresponding to a part of the curve associated with temperatures greater than or equal to the limit temperature, the limit temperature being between 500° C. and 550° C., preferentially equal to 530° C.; and
D) from the surface areas of the first and second components, the pyrogenic carbon content X₃ present in the sample is determined according to a formula of the type:

$${X}_{3} = \frac{{X}_{2} - A \cdot {X}_{1}}{1 + A \cdot B}$$

in which X₁ and X₂ are carbon contents determined respectively from the surface areas of the first and second components, A is a coefficient representing the proportion of the matrix in the second component relative to the first component, and B is a coefficient representing the proportion of the at least one of charcoal and the biochar in the first component relative to the second component.

According to an implementation of the invention, the first temperature sequence may comprise an isothermal steady stage of predetermined duration at the initial temperature (T0) of the first temperature sequence, followed by a thermal gradient to reach the final temperature (TF) of the first temperature sequence, the predetermined duration of the isothermal steady stage of the first temperature sequence possibly being between 1 and 5 minutes, and possibly preferentially equal to 3 minutes, and the thermal gradient of the first temperature sequence possibly being between 1° C./min and 50° C./min, preferentially between 15° C./min and 35° C./min, possibly being very preferentially equal to 25° C./min.

According to an implementation of the invention, the second temperature sequence may comprise at least one thermal gradient between 1° C./min and 50° C./min, preferentially between 15° C./min and 35° C./min, very preferentially equal to 25° C./min.

According to an implementation of the invention, the second temperature sequence may also comprise an isothermal steady stage of a predetermined duration at a temperature of between 49° and 600° C., preferably between 50° and 550° C., preferentially equal to 520° C., the predetermined duration of the isothermal steady stage of the second temperature sequence possibly being between 1 and 5 minutes, possibly being preferentially equal to 3 minutes.

According to an implementation of the invention, a sample of the pure organo-mineral or mineral matrix may also be available, and the coefficient A of step D) may be determined beforehand in the following manner: steps A) to C) are applied to the sample of the pure organo-mineral or mineral matrix, and the coefficient A is determined by calculating the ratio between the surface area of a second component and the surface area of a first component determined from the sample of the pure organo-mineral or mineral matrix.

According to an implementation of the invention, a sample of the at least one of pure biochar and the pure charcoal may also be available, and the coefficient B of step D) may be determined beforehand in the following manner: steps A) to C) are applied to the sample of the at least one of the biochar and the charcoal, and coefficient B is determined by calculating the ratio between the surface area of the first component and the surface area of the second component determined from the sample of the at least one of pure biochar and charcoal.

According to an implementation of the invention, step D) may be applied by use of a coefficient A ranging from a value of 0.08 to a value of 0.76, preferentially at least one of equal to 0.09, and/or a coefficient B ranging from a value of 0.90 to a value of 46.60, preferentially is equal to 20.73.

According to an implementation of the invention, a total mass of pyrogenic carbon Qc,bc_mix present in the sample may be determined according to a formula of the type: Qc,bc_mix=X3/C*K, where C is a ratio between a carbon content determined from a surface area of a second component determined for a sample of at least one of pure biochar and charcoal, and a total mass of carbon in the sample of at least one of pure biochar and charcoal, and where K is a multiplicative coefficient between 3.5 and 4.0, and preferentially is equal to 3.7.

Alternatively, a total mass of pyrogenic carbon Qc,bc_mix present in the sample may be determined according to a formula: Qc,bc_mix=X3/C*K, in which C is a ratio between a value of 0.04 and a value of 0.74, preferentially equal to 0.07, and in which K is a multiplication coefficient between 3.5 and 4.0, preferentially equal to 3.7.

Other characteristics and advantages of the process according to the invention will become apparent from reading the description below of nonlimiting implementation examples, with reference to the appended figures described below.

### BRIEF DESCRIPTION OF THE DRAWINGS

FIG. 1A schematically illustrates the evolution of temperature as a function of time in the temperature sequence of the first step of the process according to the invention.

FIG. 1B schematically illustrates the evolution of temperature as a function of time of a variant of the temperature sequence of the first step of the process according to the invention.

FIG. 2 shows, as an application example, the CO2 curve measured during step 2) of the process according to the invention, and also the first and second components resulting from the breakdown according to step 3) of the process according to the invention.

FIG. 3 schematically shows the proportion of the organo-mineral or mineral matrix and the proportion of at least one of biochar and charcoal in the first component resulting from the breakdown according to step 3) of the process according to the invention, and also the proportion of at least one of biochar and charcoal and the proportion of the organo-mineral or mineral matrix in the second component resulting from the breakdown according to step 3) of the process according to the invention.

### DETAILED DESCRIPTION OF THE INVENTION

The invention relates to a process for quantifying the pyrogenic carbon present in a sample comprising an organo-mineral or mineral matrix and also at least one of charcoal and biochar.

The term “pyrogenic carbon” means the organic fraction that has undergone pyrolysis, i.e. the effect of at least one of fire and heat (temperature >200° C.) in the absence of or in a low concentration of oxygen.

The term “organo-mineral or mineral matrix” means an unconsolidated, porous material formed of at least one of a mixture of organic and mineral particles of varying size and at least one of the chemical and mineralogical composition.

The term “charcoal” means the solid residues of a chemical transformation under the effect of a rise in temperature, resulting from pyrolysis or incomplete combustion of plant or animal biomass.

The term “biochar” means charcoal produced with the intention of using it as an organic amendment, notably to improve the physicochemical properties of a soil or its carbon storage.

The process of the invention requires at least one sample comprising an organo-mineral or mineral matrix and also at least one of charcoal and biochar.

According to an implementation of the invention, the sample comprising an organo-mineral or mineral matrix and also at least one of the charcoal and biochar may be a soil sample comprising at least one of charcoal and biochar. The term “soil” means all the external layers of the Earth's surface formations. A soil sample may be collected manually in a pit or by coring using an auger.

According to an implementation of the invention, the sample comprising an organo-mineral or mineral matrix and also at least one of the charcoal and biochar may be a sample of an amendment comprising an organo-mineral or mineral matrix and also at least one of the charcoal and biochar.

According to an implementation of the invention, the sample comprising an organo-mineral or mineral matrix and also at least one of the charcoal and biochar may be a soil sample, a sample of natural sediment or sediment polluted with fire residues, or else a sample of mineral materials (concrete, excavated earth, sediments) mixed with charcoal/biochar. The soil may be agricultural soil or anthrosol deliberately enriched with biochar.

Advantageously, the sample may be sieved using a sieve with orifices having a diameter of 2 mm, dried at a temperature below 40° C., and then ground until fragments having dimensions of less than 200 μm are obtained.

Preferably, a sample of the pure organo-mineral or mineral matrix, i.e. comprising neither biochar nor charcoal, is also available.

Advantageously, a sample of the at least one of the biochar and charcoal present in the sample under consideration may also be available.

The process according to the invention may be advantageously but non-limitingly performed using the Rock-Eval® device (IFP Energies Nouvelles, France), as described in patents FR 2 227 797 (U.S. Pat. No. 3,953,171) and FR 2 472 754 (U.S. Pat. No. 4,352,673). Specifically, the Rock-Eval® device comprises at least:
a furnace for pyrolysis in a non-oxidizing atmosphere;
means for transferring the pyrolysis residues into an oxidation furnace;
a furnace for oxidation in an oxidizing atmosphere;
means for measuring the amount of hydrocarbon (HC) compounds released during the pyrolysis; and
means for measuring the carbon monoxide (CO) and the carbon dioxide (CO₂).

The process may also be performed using a single pyrolysis furnace, which can operate both in a non-oxidizing atmosphere and in an oxidizing atmosphere, interacting with a device for measuring the amount of hydrocarbon compounds released during the pyrolysis and a device for measuring the carbon monoxide and the carbon dioxide.

The process according to the invention comprises at least the following steps:
1) Heating sequence under an inert atmosphere (pyrolysis)
2) Heating sequence under an oxidizing atmosphere (oxidation)
3) Decomposition into two components
4) Determination of the pyrogenic carbon content

The steps of the process according to the invention are detailed below.

### 1. Heating Sequence Under an Inert Atmosphere (Pyrolysis)

During this step, the sample comprising an organo-mineral or mineral matrix and also at least one of the biochar and charcoal is heated under an inert atmosphere (for instance a nitrogen or helium stream) according to a temperature sequence with an initial temperature (noted TO hereinbelow) of between 10° and 300° C., preferentially equal to 200° C., and a final temperature (noted TF hereinbelow) of between 50° and 800° C., preferentially equal to 650° C.

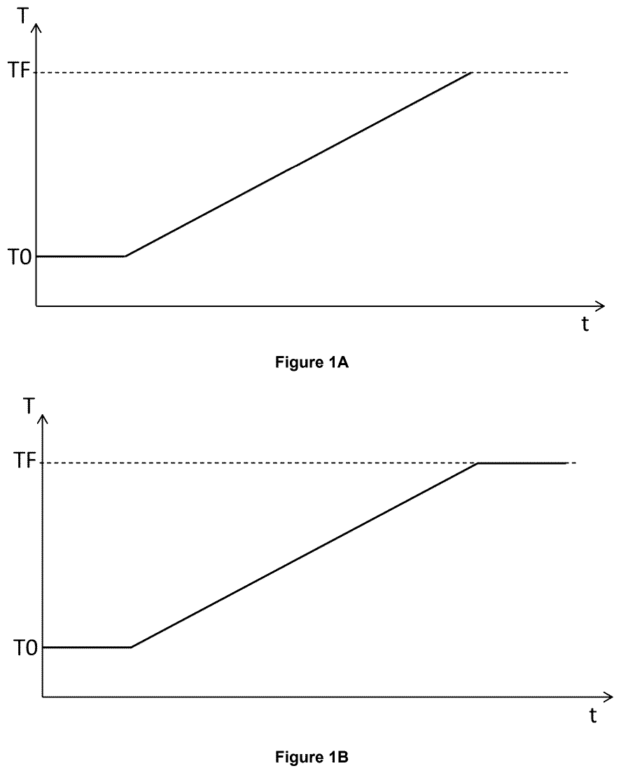

In a preferred manner, the temperature sequence under an inert atmosphere may comprise at least one isothermal steady stage at the initial temperature TO, followed by a predetermined thermal gradient so as to raise the sample temperature to the final temperature TF. FIG. 1A diagrammatically illustrates the change in the temperature T as a function of the time t of such a sequence of temperatures, having an isothermal steady stage at the temperature TO, followed by a thermal gradient up to the temperature TF.

Advantageously, the temperature sequence under an inert atmosphere of this embodiment may also comprise a second isothermal steady stage, at the final temperature TF. In other words, a second isothermal steady stage at the final temperature TF follows the phase of the temperature sequence appearing in the form of a thermal gradient. This makes it possible to continue, if necessary, the cracking of the compounds having a cracking temperature close to the final temperature TF of the temperature sequence under an inert atmosphere according to the invention. FIG. 1B schematically illustrates the change in the temperature T as a function of the time t of such a temperature sequence, having two isothermal steady stages, at the temperatures TO and TF as defined above, connected together by a thermal gradient.

According to an implementation of the invention, the initial temperature TO is preferably equal to 200° C. This temperature is indeed sufficient to release the most labile organic compounds present in most soil, organic amendment or sediment samples.

According to an implementation of the invention, the final temperature TF is preferably equal to 650° C., so as to avoid obtaining CO and CO₂ curves with incomplete peaks at the end of pyrolysis, measured notably on natural samples (fresh and dried plant tissues, litter, peat and plant composts, organo-mineral and mineral soils, surface formations).

According to an implementation of the invention, the isothermal steady stage(s) of the temperature sequence under an inert atmosphere may have a nonzero predetermined duration (for example, greater than half a minute), preferentially between 1 and 5 minutes, and very preferentially equal to 3 minutes. Such durations make it possible to regard the cracking of the compounds having a cracking temperature close to the temperature of the isothermal steady stage as being complete. According to the implementation of the invention according to which the temperature sequence under an inert atmosphere according to the invention comprises several isothermal steady stages and in particular two isothermal steady stages at the temperatures TO and TF, the duration of one isothermal steady stage can be different from the duration of the other isothermal steady stage(s).

According to an implementation of the invention, the thermal gradient(s) of the temperature sequence under an inert atmosphere may be between 1 and 50° C./min, preferably between 15° and 35° C./min, and are preferably equal to 25° C./min. Such values constitute compromises allowing thermal cracking of the compounds, while at the same time limiting the duration of implementation of the process.

According to an implementation of the invention, it is possible to measure, continuously (i.e. continuously over time), an amount of hydrocarbon compounds released during heating under at least one of an inert atmosphere, and an amount of CO₂ and at least one of an amount of CO contained in an effluent resulting from the heating. In other words, during this sequence, the amount of HC, of CO and of CO₂ which are released by the sample by thermal cracking of the organic matter and by the thermal decomposition of the carbonate-based minerals can be continuously measured. The amount of hydrocarbon compounds can be measured by use of a detector of the flame ionization (FID) type. The amount of CO and of CO₂ which are released can be measured by a detector of the infrared (IR) type. As a variant, other devices for measuring the amount of HC, of CO and/or of CO₂ can be used. According to this implementation, on conclusion of this step applied to a given sample, a first curve representative of the amount of hydrocarbon compounds which are released over time during the pyrolysis phase is obtained, along with two other curves representative of the amount of CO and CO₂ which are released over time during the pyrolysis phase. Such measurements can help to determine standard parameters for such a thermal analysis, in particular the parameter denoted TOC (Total Organic Carbon), which corresponds to the carbon content of the sample, determined from the amount of hydrocarbons released by the sample and from the amounts of CO and of CO₂ released below threshold temperatures during the pyrolysis phase and the oxidation phase, and the parameter denoted MinC (Mineral Carbon), which corresponds to the mineral carbon content of the sample, determined from the amounts of CO and of CO₂ released by the sample above threshold temperatures during the pyrolysis phase and the oxidation phase. A description of these general parameters can be found in the document (Behar et al., 2001).

Generally, this particular heating sequence under an inert atmosphere is sufficient to allow the thermal cracking of classes of compounds comprising mineral carbon and organic carbon, notably:
thermally very labile compounds, which are particularly abundant in fresh biological tissues and which are generally released at temperatures of between approximately 80° C. and 360° C.;
thermally labile compounds, which are predominant in organic samples, such as litter or peat, and which are generally released at temperatures of between approximately 360° C. and 420° C.;
thermally resistant compounds, which are predominant in organo-mineral samples (soils) or mineral samples (alluvial deposits, colluvial deposits) and which are generally released at temperatures of between approximately 420° C. and 470° C.;
thermally refractory compounds, which are generally released at temperatures of between approximately 470° C. and 520° C.;
and thermally very refractory compounds, which are present in greater proportions in decomposition residues or exogenous fractions, such as pyrogenic or petrogenic organic matter, and which are generally released at temperatures of between approximately 520° C. and 650° C.

According to an implementation of the invention, the temperature sequence under an inert atmosphere according to the invention can be preceded by a phase of rise in temperature of the pyrolysis furnace, which can be in the form of a thermal gradient, for example between 1 and 50° C./min, preferably between 2° and 25° C./min, or of any other form of curve of rise in temperature of the pyrolysis furnace. This preliminary phase of rise in temperature of the pyrolysis furnace makes it possible to bring the pyrolysis furnace to the initial temperature of the temperature sequence under an inert atmosphere according to the invention. This preliminary phase can contribute toward starting the thermal cracking of the compounds, the cracking temperature of which is less than the initial temperature of the temperature sequence under an inert atmosphere according to the invention, notably in the case of fresh biological tissues.

According to an implementation of the invention, the temperature sequence under an inert atmosphere according to the invention can be followed by a phase of lowering in temperature of the pyrolysis furnace, which can be in the form of a thermal gradient, for example between −1 and −50° C./min, preferably between −20 and −25° C./min, or of any other form of curve of lowering in temperature of the pyrolysis furnace. This terminal phase of lowering in temperature of the pyrolysis furnace makes it possible, if necessary, to complete the thermal cracking of the compounds which are associated with the final temperature of the temperature sequence under an inert atmosphere according to the invention.

### 2) Heating Sequence Under an Oxidizing Atmosphere (Oxidation)

In this second step, the solid residue of the sample obtained on conclusion of the sequence of heating under an inert atmosphere as described in step 1 above is subjected to oxidation according to a predefined temperature sequence, with an initial temperature (referred to hereinbelow as TO′) of between 10° and 300° C., preferentially equal to 200° C., and a final temperature (referred to hereinbelow as TF′) of between 700 and 1000° C., preferably equal to 850° C. (so as to deplete the mineral carbon stock).

According to an implementation of the invention, the temperature sequence of this heating under an oxidizing atmosphere comprises at least one thermal gradient of between 1 and 50° C./min, preferably between 15° and 35° C./min, preferably equal to 25° C./min. This second temperature sequence may notably comprise such a thermal gradient to link the initial TO′ and final TF′ temperatures of the temperature sequence under an oxidizing atmosphere.

In general, the preferential temperature range for the initial temperature TO′ of the temperature sequence under an oxidizing atmosphere helps to avoid episodes of instantaneous combustion of the sample residue at the start of the oxidation cycle.

According to an implementation of the invention, the temperature sequence under an oxidizing atmosphere can also comprise an isothermal steady stage at the initial temperature TO′ of a predetermined nonzero duration (for example greater than half a minute), and may preferentially be between 1 and 5 minutes, very preferentially 3 minutes.

According to the invention, at least an amount of CO₂ (and optionally an amount of CO) released during this second temperature sequence is measured continuously. According to an implementation of the invention, this measurement may be performed using an infrared (IR) sensor. It should be noted that such a sensor provides values measured in millivolts (mV). In a conventional manner, an amount of CO₂ released during this second temperature sequence, noted as Xtot hereinbelow, is determined by determining an area under the curve measured (optionally between predefined temperatures) by this sensor, according to a formula:

$$Xtot = \frac{12}{44.} . \frac{SurfC}{mass} \qquad ( 1 )$$

in which SurfC corresponds to the area under the curve (also known as the thermogram) representing the amount of CO₂ released during this second temperature sequence, mass corresponds to the mass of the sample, and in which Xtot is expressed as mg/g of sample. As a variant, other devices of measuring the amount of CO₂ may be used.

Advantageously, the temperature sequence under an oxidizing atmosphere may also comprise an isothermal steady stage of predetermined duration at a temperature of between 49° and 600° C., preferably between 50° and 550° C., preferentially equal to 520° C. This isothermal steady stage makes it possible to better separate, in a curve representing the temperature-dependent evolution of the amount of CO₂ released during heating under an oxidizing atmosphere, a component attributed to biochar or charcoal from a component attributed to the mineral or organo-mineral matrix present in the sample under consideration. This notably allows the result of step 3) of the process according to the invention described below to be improved. This isothermal steady stage may be of a predetermined nonzero duration (for example greater than half a minute), preferentially between 1 and 5 minutes, very preferentially 3 minutes. According to this embodiment, the temperature sequence under an oxidizing atmosphere may comprise two thermal gradients, preferably between 1 and 50° C./min, more preferably between 15 and 35° C./min, very preferentially equal to 25° C./min. According to this implementation, the isothermal steady stage at a temperature of between 490 and 600° C., preferably between 50° and 550° C., preferentially equal to 520° C., may be both preceded and followed by the at least two thermal gradients of this embodiment.

3) Breakdown into Two Components

During this step, a curve representing the temperature-dependent evolution of the amount of CO₂ released during the second heating sequence is broken down into at least a first and a second component, the first component corresponding to the part of the curve associated with temperatures below a limit temperature, and the second component corresponding to the part of the curve associated with temperatures greater than or equal to the limit temperature, limit temperature being between 500° C. and 550° C., preferentially equal to 530° C.

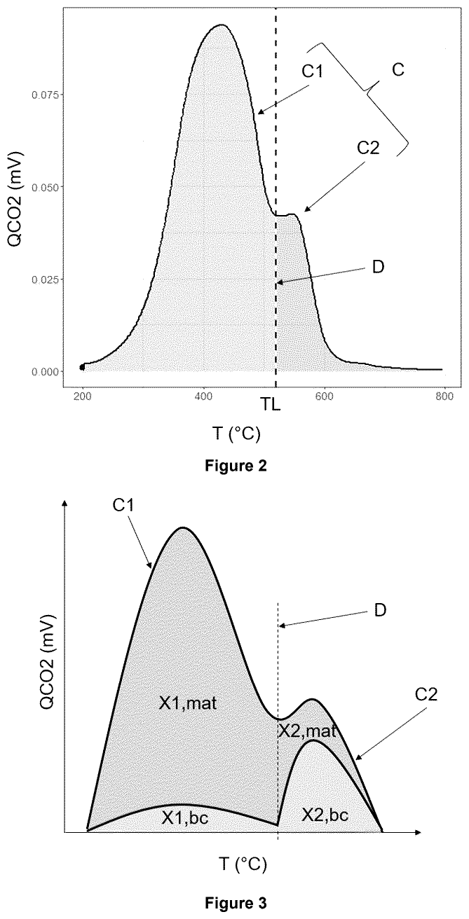

In other words, during this step, two components are delimited on the CO₂ curve measured in step 2) as a function of a vertical line passing through the limit temperature according to the invention. This is illustrated in FIG. 2, which shows the result of breaking down a curve C measuring the amount of CO₂ QCO2 released during step 2) of the process according to the invention into two components C1, C2 as a function of a vertical line D passing through the limit temperature LT, 520° C. for this illustration.

The limit temperature according to the invention corresponds to the temperature below which most of the CO₂ from the mineral or organo-mineral matrix is released, and above which most of the CO₂ from the biochar or charcoal is released. This limit temperature was able to be observed on several soil or sediment samples comprising biochar or charcoal. A first component is thus obtained, representing the proportion of CO₂ released predominantly by the mineral or organo-mineral matrix making up the sample under consideration, and a second component representing the proportion of CO₂ released predominantly by the at least one of biochar and charcoal making up the sample under consideration.

According to an implementation of the invention, the limit temperature according to the invention may be determined by determining the temperature of an inflection point of the curve representing the evolution as a function of temperature of the amount of CO₂ released during the second heating sequence in a part of this curve between 500° C. and 550° C.
4) Determination of the pyrogenic carbon content In this step, the pyrogenic carbon content in the sample under consideration is determined from the surface areas of the first and second components determined in step 3). More specifically, according to the invention, the pyrogenic carbon content present in the sample under consideration is determined, noted as X₃ hereinbelow, according to a formula of the type:

$${X}_{3} = \frac{{X}_{2} - A \cdot {X}_{1}}{1 + A \cdot B} \qquad ( 2 )$$

in which X₁ and X₂ are carbon contents determined respectively from the surface areas of the first and second components, A is a coefficient representing the proportion of the matrix in the second component relative to the first component, and B is a coefficient representing the proportion of the charcoal and/or the biochar in the first component relative to the second component.

According to an implementation of the invention, X₁ can be determined according to the formula:

$${X}_{1} = SurfG 1 \qquad ( 3 )$$

and X₂ can be determined according to the formula:

$${X}_{2} = SurfG 2 \qquad ( 4 )$$

in which SurfG1 and SurfG2 are, respectively, the surface areas of the first and second components determined on conclusion of step 3 of the process according to the invention.

Equation (2) arises from the fact that the breakdown according to step 3) is imperfect for separating the contribution of the organo-mineral or mineral matrix from the contribution of at least one of the biochar and charcoal in a curve measuring the amount of CO₂ released during an oxidation phase. In other words, the first component resulting from the breakdown according to the invention does indeed comprise a predominant contribution linked to the organo-mineral or mineral matrix (noted as X_{1,mat} hereinbelow), but also includes a contribution from biochar and/or charcoal (noted as X_{1,bc} hereinbelow). Similarly, the second component resulting from the breakdown according to the invention does indeed comprise a predominant contribution from at least one of the biochar and charcoal (noted as X_{2,bc} hereinbelow), but also includes a contribution linked to the organo-mineral or mineral matrix (noted as X1,bc hereinbelow). This is notably illustrated in FIG. 3, which schematically represents the proportion of the organo-mineral or mineral matrix X1,mat and the proportion of at least one of the biochar and charcoal X1,bc in the first component C1, and also the proportion of at least one of the biochar and charcoal X2,bc and the proportion of the organo-mineral or the mineral matrix X2,mat in the second component C2, which the components C1 and C2 being delimited by the line D. With these notations, the coefficients A and B according to the invention may be described by the following formula:

$$A = \frac{{X}_{2 , mat}}{{X}_{1 , mat}} \qquad ( 5 )$$

$$and$$

$$B = \frac{{X}_{1 , bc}}{{X}_{2 , bc}} \qquad ( 6 )$$

According to a first variant of the invention, at least one of the coefficient A and coefficient B may be determined from a sample of the pure organo-mineral or mineral matrix and a sample of at least one of the pure biochar and charcoal respectively, representative of the organo-mineral or mineral matrix and of at least one of the biochar and charcoal present in the sample under consideration, to which steps 1), 2) and 3) described above are applied. Coefficient A may thus be determined by the ratio between the surface area of the second component and the surface area of the first component determined from the sample of pure organo-mineral or mineral matrix. Coefficient B may be determined by the ratio between the surface area of the first component and the surface area of the second component determined from at least one of the pure biochar and charcoal sample.

According to a second variant of the invention, and notably if samples of the pure organo-mineral or mineral matrix and at least one of the pure biochar and charcoal, representative of the organo-mineral or mineral matrix and at least one of the biochar and charcoal present in the sample under consideration, are not available, equation (2) above can be used with a coefficient A between a value of 0.08 and a value of 0.76, preferentially at least one of equal to 0.09, and a coefficient B between a value of 0.90 and a value of 46.60, preferentially equal to 20.73. These ranges and preferential values of coefficients A and B were determined from a plurality of samples of pure organo-mineral or mineral matrix and a plurality of samples of at least one of the pure biochar and charcoal, of different types, to which the method described above was applied. In particular, samples of pure organo-mineral or mineral matrix of soil and sediment type from various climatic conditions and with various total organic carbon contents, and samples of at least one of the pure biochar and charcoal of various plant biomass and various pyrolysis temperatures, between 450° C. and 650° C., were used. The preferential value of coefficients A and B corresponds to the median of the values thus determined for the plurality of samples.

Thus, on conclusion of this step, the pyrogenic carbon content present in the sample under consideration, comprising both a mineral or organo-mineral matrix and at least one of the charcoal and biochar, is obtained.

According to an implementation of the invention, the total mass of pyrogenic carbon present in the sample under consideration, noted as Qc,bc_mix hereinbelow, may be determined according to a formula of the type:

$$Q c , bc\_mix = X 3 / C * K \qquad ( 7 )$$

in which
C is the ratio between the carbon content determined from the surface area of the second component determined in the case of a sample of at least one of the pure biochar and charcoal, noted as X2,bc, and the total mass of carbon in the sample of at least one of the pure biochar and charcoal; in other words, the ratio C can be written as follows:

$$C = X 2 , bc / ( TOC , bc * Qty , bc ) \qquad ( 8 )$$

in which TOC,bc and Qty,bc respectively correspond to the total organic carbon and the total mass of a pure biochar/charcoal sample. According to an implementation of the invention, and notably if at least one of a pure biochar and charcoal sample, representative of at least one of the biochar and charcoal present in the sample under consideration, is not available, equation (2) above can be used by using a ratio C between a value of 0.04 and a value of 0.74, preferentially equal to 0.07. This range and this preferential value were determined from a plurality of different types of at least one of the pure biochar and charcoal samples. The preferential value corresponds to the median of the values thus determined for the plurality of samples.
K is a multiplicative coefficient. According to an implementation of the invention, the coefficient K may be between 3.5 and 4.0, and is preferentially equal to 3.7. Such values were determined from a plurality of different types of pure biochar and/or charcoal samples.

### EXAMPLES

The characteristics and advantages of the process according to the invention will become more clearly apparent on reading the application example below.

The present invention is applied to determine the mass of pyrogenic carbon present in a sample corresponding to a soil-biochar mixture.

A plurality of samples are generated, for different mass ratios between biochar and soil, by homogeneous mixing between a soil of agricultural origin, and an industrial biochar of herbaceous plants. The total organic carbon (TOC) of the biochar and the soil is 82.60% and 5.55%, respectively. Each sample thus formed is dried at a temperature of less than or equal to 40° C. until its weight has stabilized, and is then ground to below 200 μm.

Each sample is heated under an inert atmosphere according to the invention, and its residue is then heated under an oxidizing atmosphere according to the invention. FIG. 2 shows, in the case of a sample containing 1% by mass of biochar, curve C of the amount of CO₂ QCO2 measured during step 2) of the process according to the invention, and also the result of step 3) of the process according to the invention, in the form of two components C1, C2 delimited by the vertical line D passing through the limit temperature LT equal to 520° C.

Table 1 shows the mass of pyrogenic carbon (given in mg of carbon, mgC) present in the samples under consideration, determined on conclusion of step 4 of the process according to the invention applied according to the first variant described above (determination of coefficients A and B of equation (2) from pure soil and biochar samples; column Qc,bc_mix_V1) and according to the second variant described above (determination of coefficients A and B of equation (2) from their preferred values defined above; column Qc,bc_mix_V2), and also the actual mass of pyrogenic carbon present in the samples under consideration (column Qc,bc_mix_REAL), as a function of their mass ratio between biochar and soil (Ratio column). It can be seen that the mass of pyrogenic carbon determined by the present invention, applied according to its first variant or its second variant, are very close to the actual values (average error of 1.36% and maximum of 26.37% for the first variant; average error of −18.43% and maximum of −46.80% for the second variant).

These results were obtained in less than 90 minutes for each sample, which essentially corresponds to the heating time under an inert atmosphere and the heating time under an oxidizing atmosphere for each sample.

Thus, the present invention makes it possible to quickly and accurately quantify the pyrogenic carbon in a sample of an organo-mineral or mineral matrix also comprising at least one of the biochar and charcoal, by means of a thermal analysis that is simple to perform.

| | Qc,bc_mix REAL | Qc,bc_mix V1 | Qc,bc_mix_V2 |
|---|---|---|---|
| Ratio | (mgC) | (mgC) | (mgC) |
| 0.05 | 4.5 | 5.7 | 2.4 |
| 0.11 | 9.0 | 9.4 | 9.7 |
| 0.21 | 17.3 | 17.3 | 17.7 |
| 0.51 | 43.2 | 39.3 | 40.2 |
| 0.56 | 47.3 | 42.3 | 38.8 |
| 1.02 | 84.9 | 78.9 | 76.0 |
| 1.11 | 93.0 | 97.8 | 100.1 |

1-9. (canceled)

10. A process for quantifying pyrogenic carbon content present in a sample of an organo-mineral or a mineral matrix and at least one of charcoal and biochar, comprising:
A) heating the sample under an inert atmosphere according to a first temperature sequence, with an initial temperature of between 100 and 300° C., and a final temperature of between 50° and 800° C.;
B) heating a residue of the sample resulting from the heating under an inert atmosphere, under an oxidizing atmosphere according to a second temperature sequence with an initial temperature of between 10° and 300° C., and a final temperature of between 70° and 1000° C., and measuring at least one amount of CO₂ released during the second temperature sequence;
C) based on a curve representing a temperature-dependent evolution of the quantity of CO₂ released during the heating in an oxidizing atmosphere, breaking the curve down into at least a first and a second component, the first component corresponding to a part of the curve associated with temperatures below a temperature limit, and the second component corresponding to a part of the curve associated with temperatures equal to the temperature limit or greater than the temperature limit wherein the temperature limit is between 500° C. and 550° C.; and
D) determining the pyrogenic carbon X₃ present in the sample resulting from the surface areas of the first and second components, according to a formula:
$${X}_{3} = \frac{{X}_{2} - A \cdot {X}_{1}}{1 + A \cdot B}$$
in which X₁ and X₂ are carbon contents determined respectively from surface areas of the first and second components, A is a coefficient representing a proportion of the matrix in the second component relative to the first component, and B is a coefficient representing a proportion of the at least one of the charcoal and the biochar in the first component relative to the second component.

11. The process as claimed in claim 10, wherein the first temperature sequence comprises an isothermal steady stage of predetermined duration at the initial temperature of the first temperature sequence, followed by a thermal gradient to reach the final temperature of the first temperature sequence, the predetermined duration of the isothermal steady stage of the first temperature sequence being between 1 and 5 minutes, and the thermal gradient of the first temperature sequence being between 1° C./min and 50° C./min.

12. The process as claimed in claim 11, wherein the first temperature sequence is between 15° C./min and 35° C./min.

13. The process as claimed in claim 10, in which the second temperature sequence comprises at least one thermal gradient between 1° C./min and 50° C./min.

14. The process as claimed in claim 12, wherein the at least one thermal gradient is between 15° C./min and 35° C./min.

15. The process as claimed in claim 13, wherein the at least one thermal gradient is between 15° C./min and 35° C./min.

16. The process as claimed in claim 10, wherein the second temperature sequence also comprises an isothermal steady stage of a predetermined duration at a temperature of between 490 and 600° C., the predetermined duration of the isothermal steady stage of the second temperature sequence is between 1 and 5 minutes.

17. The process as claimed in claim 10, wherein a sample of the pure organo-mineral or pure mineral matrix is present, and the coefficient A of step D) is determined beforehand in which steps A) to C) are applied to the sample of the pure organo-mineral or mineral matrix, and the coefficient A is determined by calculating a ratio between a surface area of a second component and the surface area of a first component which D) is determined from the sample of the pure organo-mineral or pure mineral matrix.

18. The process as claimed in claim 10, wherein a sample of at least one of the pure biochar and the pure charcoal is present, and the coefficient B of step D) is determined beforehand by applying steps A) to C) to the sample of at least one of the pure biochar and the pure charcoal, and coefficient B is determined by calculating a ratio between surface area of the first component and surface area of the second component is determined from the sample of at least one of the pure biochar and pure charcoal.

19. The process as claimed in claim 10, wherein step D) is applied with coefficient A ranging from a value of 0.08 to a value of 0.76, and a coefficient B is applied ranging from a value of 0.90 to a value of 46.60.

20. The process as claimed in claim 10, wherein a total mass of pyrogenic carbon Qc,bc_mix in the sample is determined according to a formula: Qc,bc_mix=X3/C*K, wherein C is a ratio between carbon content determined from a surface area of a second component determined for a sample of at least one of the pure biochar and the pure charcoal, and a total mass of carbon in the sample of at least one of the pure biochar and the charcoal, and where K is a multiplicative coefficient between 3.5 and 4.0.

21. The process as claimed in claim 10, wherein a total mass of pyrogenic carbon Qc,bc_mix is present in the sample which is determined according to a formula: Qc,bc_mix=X3/C*K, wherein C is a ratio between 0.04 and 0.74, and K is a multiplication coefficient between 3.5 and 4.0.
